(19) United States
(12) Patent Application Publication
(10) Pub. No.: US 2024/0082184 A1
(43) Pub. Date: Mar. 14, 2024

(54) IMPROVED TREATMENT FOR GLOBOID CELL LEUKODSYTROPHY OR KRABBE DISEASE

(71) Applicant: Rush University Medical Center, Chicago, IL (US)

(72) Inventor: Kalipada Pahan, Skokie, IL (US)

(21) Appl. No.: 18/261,959

(22) PCT Filed: Jan. 20, 2021
(86) PCT No.: PCT/US2021/014124

§ 371 Date: Jul. 18, 2023

(51) Int. Cl. A61K 31/192; A61K 31/07; A61P 25/28

(52) U.S. Cl. CPC A61K 31/192; A61K 31/07; A61P 25/28

(57) ABSTRACT

The present disclosure generally relates to improved pharmaceutical compositions useful for the treatment of diseases and disorders. More particularly, the disclosure relates to pharmaceutical compositions comprising gemfibrozil and/or cinnamic acid for the treatment of globoid cell leukodystrophy or Krabbe disease.

### FIELD OF THE INVENTION

The present disclosure generally relates to improved pharmaceutical compositions useful for the treatment of diseases and disorders. More particularly, the disclosure relates to pharmaceutical compositions comprising gemfibrozil and/or cinnamic acid for the treatment of globoid cell leukodystrophy or Krabbe disease.

### BACKGROUND OF THE INVENTION

Globoid cell leukodystrophy or Krabbe disease (KD) is an inborn error of metabolism. It is caused due by deficiency of the lysosomal enzyme galactocerebrosidase (GALC). Normally, the function of GALC is to catabolize cytotoxic lipid galactosylsphingosine or psychosine. When there is a lack of GALC, this cytotoxic lipid galactosylsphingosine or psychosine accumulates in the central nervous system (CNS) and peripheral nervous system (PNS), ultimately leading to a dysmyelinating phenotype in KD. As a result, KD is a devastating illness that typically leads to the death of affected children within the first two years of life.

The only currently available treatment for KD is hematopoietic stem-cell transplantation. However, this complicated treatment exhibits only modest improvements and only if begun before or early in disease onset. See Krivit et al., “Hematopoietic stem-cell transplantation in globoid-cell leukodystrophy,” N. Engl J Med. (1998) 338(16): pp. 1119-26.

Cinnamon, the brown bark of cinnamon tree, is a commonly used spice and flavoring material for dessert, candies, chocolate etc. It has a long history of being used as medicine as well. Medieval physicians used cinnamon in medicines to treat a variety of disorders, including arthritis, coughing, hoarseness, sore throats, etc. In addition to containing manganese, dietary fiber, iron, and calcium, cinnamon contains three major compounds—cinnamaldehyde, cinnamyl acetate and cinnamyl alcohol. After intake, these three active compounds are converted into cinnamic acid by oxidation and hydrolysis, respectively. Then, cinnamic acid is β-oxidized to benzoate in the liver. This benzoate exists as sodium salt (sodium benzoate) or benzoyl-CoA.

In previous studies, the current inventors have demonstrated that cinnamic acid stimulates suppressor of cytokine signaling 3 (SOCS3) to inhibit the activation of microglia. See Chakrabarti et al., (2018) Curr Alzheimer Res 15, 894-904. It was further found that oral administration of cinnamic acid protects mice from Alzheimer's disease (17) and Parkinson's disease (25). See Chandra et al., (2019) Neurobiol Dis 124, 379-395; Prorok et al., (2019) Neurochem Res 44, 751-762.

Sodium benzoate is a widely-used food preservative due to its anti-microbial properties. It also has medical importance as a component of UCEPHAN®, a Food and Drug Administration (FDA)-approved drug (now discontinued) used in the treatment for hepatic metabolic defects associated with hyperammonemia, such as urea cycle disorder. The present inventor explored a novel use of sodium benzoate in treating the disease process of relapsing-remitting EAE in female SJL/J mice (see Brahmachari et al., J. Immunol., 2007, 179(1):275-83, the entire contents of which are expressly incorporated into the present application by reference).

The present inventor also discovered that sodium benzoate suppresses the disease process of multiple sclerosis (MS) in mice. The inventor has also discovered that sodium benzoate up-regulates a protein called DJ-1, which is a beneficial, neuroprotective protein having implications in neurodegenerative disorders, such as Parkinson's disease (PD) and Alzheimer's disease (AD) (see Khasnavis et al., Journal of Neuroimmune Pharmacology, 2012, 7: pp 424-435, the entire contents of which are expressly incorporated into the present application by reference).

Further, it has been found that the level of neurotrophic factors, such as brain-derived neurotrophic factor (BDNF) and neurotrophin-3 (NT-3), decreases in the brain of patients with different neurodegenerative disorders, such as AD and PD. Recently, the present inventor delineated that sodium benzoate increases the production of BDNF and NT-3 in brain cells, indicating that it could be beneficial for neurodegenerative disorders (see Jana et al., J. Neuroimmune Pharmacol., 2013, 8(3):739-55, the entire contents of which are expressly incorporated into the present application by reference).

However, sodium benzoate is quickly metabolized and excreted from the body. Therefore, sodium benzoate is generally administered multiple times per day in order to ensure continual removal of toxic ammonia from the bloodstream.

Gemfibrozil, a fibrate drug, is commonly known as “Lopid” in the pharmacy. In the year of 1976, gemfibrozil was successfully introduced in the market as a lipid-lowering drug with its profound ability to reduce the level of plasma triglyceride. In addition to its hypolipidemic effect, we and others have seen that gemfibrozil can also regulate many other signaling pathways responsible for inflammation, switching of T-helper cells, cell-to-cell contact, migration, oxidative stress, myelination, synthesis of trophic factors, memory and learning, etc. See for example: Roy et al., (2013) Cell Rep 4, 724-737; Roy et al., (2015) Cell Metab 22, 253-265; Jana et al., (2012) J Biol Chem 287, 34134-34148; Jana et al., (2012) Neurochem Res 37, 1718-1729; Pahan et al., (2002) J Biol Chem 277, 45984-45991; Dasgupta et al., (2007) Mol Pharmacol 72, 934-946; Roy et al., (2007) J Biol Chem 282, 32222-32232; Roy et al., (2013) J Clin Cell Immunol 7, 158.

The inventors have further found that gemfibrozil as well as the combination of gemfibrozil and vitamin A derivative retinoic acid upregulate Cln2/TPP1 and stimulate lysosomal biogenesis in the brain and brain cells. See Ghosh et al., (2012) J Biol Chem 287, 38922-38935; Ghosh et al., (2015) J Biol Chem 290, 10309-10324; Ghosh et al; (2017) J Neurochem 141, 423-435.

U.S. Patent Publication No. 20190358188 discloses a method of decreasing neuronal apoptotic cell death in a subject having a neuro-degenerative disease by the administration of a fibrate such as gemfibrozil. U.S. Pat. No. 9,750,712 discloses a method for treatment of neuronal ceroid lipofuscinosis with a therapeutically effective amount of an agent that mediates upregulation of TPP1 such as gemfibrozil. U.S. Pat. No. 10,357,471 discloses a method for treating of neuronal ceroid lipofuscinosis by the administration of all trans retinoic acid or vitamin A. U.S. Patent Publication No. 20190336465 discloses a method for treating a neurodegenerative disease such as Alzheimer's disease, Huntington's disease, Amyotrophic lateral sclerosis (ALS), Parkinson's disease, Tay-Sach's disease or Niemann-Pick disease with gemfibrozil in combination with all-trans retinoic acid.

However, what is needed is are compositions and methods for new and effective therapeutic regimens for KD is of the highest importance. Specifically, what is needed are drugs that target and correct, reverse or ameliorate the neurochemical abnormalities seen in KD to change the course of this devastating disease.

### SUMMARY OF THE INVENTION

The inventor has discovered compositions and methods for treating various disorders and diseases, particularly, globoid cell leukodystrophy or Krabbe disease (KD). Specifically, the inventor has discovered that administration of oral gemfibrozil alone, the combination of gemfibrozil and vitamin A or cinnamic acid alone were capable of protecting myelin, suppressing glial inflammation, improving locomotor activities, and increasing lifespan in GALC^{-/-} mice which is a mouse model of Krabbe disease.

In some embodiments, the present disclosure provides a method for protecting myelin and inhibiting the progression of KD. The method comprises administering to a patient in need thereof an effective amount of an oral pharmaceutical composition comprising gemfibrozil or a combination of gemfibrozil and vitamin A.

In other embodiments, methods are provided for suppressing or inhibiting the glial inflammation associated with KD and thus treating or inhibiting the progression of the disease. Such methods comprise administering to a patient in need thereof an effective amount of an oral pharmaceutical composition comprising gemfibrozil or a combination of gemfibrozil and vitamin A.

In yet other embodiments, methods are provided for improving the locomotor activities associated with KD and thus treating or inhibiting the progression of the disease. Such methods comprise administering to a patient in need thereof an effective amount of an oral pharmaceutical composition comprising gemfibrozil or a combination of gemfibrozil and vitamin A.

In still other embodiments, methods are provided for increasing the lifespan of an individual afflicted with KD and thus treating or inhibiting the progression of the disease. Such methods comprise administering to a patient in need thereof an effective amount of an oral pharmaceutical composition comprising gemfibrozil or a combination of gemfibrozil and vitamin A.

In any of the disclosed embodiments, the pharmaceutical composition may be administered to the patient in any including one time per day, two times per day, and three times per day.

The present disclosure also provides a method for inhibiting progression of a neurodegenerative disorder wherein the neurodegenerative disorder is KD. The method comprises administering to a patient in need thereof an effective amount of a pharmaceutical composition comprising gemfibrozil alone or gemfibrozil in combination with vitamin A.

In some embodiments, the locomotor activity that is improved by administration of gemfibrozil alone or gemfibrozil in combination with vitamin A may include one or more of walking, running, jumping, and any combination thereof.

In other embodiments, the present disclosure provides a method for protecting myelin and inhibiting the progression of KD. The method comprises administering to a patient in need thereof an effective amount of an oral pharmaceutical composition comprising cinnamic acid.

In other embodiments, methods are provided for suppressing or inhibiting the glial inflammation associated with KD and thus treating or inhibiting the progression of the disease. Such methods comprise administering to a patient in need thereof an effective amount of an oral pharmaceutical composition comprising cinnamic acid.

In yet other embodiments, methods are provided for improving the locomotor activities associated with KD and thus treating or inhibiting the progression of the disease. Such methods comprise administering to a patient in need thereof an effective amount of an oral pharmaceutical composition comprising cinnamic acid.

In still other embodiments, methods are provided for increasing the lifespan of an individual afflicted with KD and thus treating or inhibiting the progression of the disease. Such methods comprise administering to a patient in need thereof an effective amount of an oral pharmaceutical composition comprising cinnamic acid.

In any of the disclosed embodiments, the pharmaceutical composition comprising cinnamic acid may be administered to the patient in any including one time per day, two times per day, and three times per day.

The present disclosure also provides a method for inhibiting progression of a neurodegenerative disorder wherein the neurodegenerative disorder is KD. The method comprises administering to a patient in need thereof an effective amount of a pharmaceutical composition comprising cinnamic acid.

In some embodiments, the locomotor activity that is improved by administration of cinnamic acid may include one or more of walking, running, jumping, and any combination thereof.

In any of the embodiments, the present disclosure also provides a method comprises administering to a patient in need thereof an effective amount of a pharmaceutical composition comprising gemfibrozil alone or gemfibrozil and vitamin A wherein the administering comprises buccal or sublingual administration.

In any of the embodiments, the present disclosure also provides a method comprises administering to a patient in need thereof an effective amount of a pharmaceutical composition comprising cinnamic acid wherein the administering comprises buccal or sublingual administration.

The present disclosure further provides an oral gemfibrozil and/or gemfibrozil and vitamin A dosage form, which comprises gemfibrozil or gemfibrozil and vitamin A in buccal, sublingual, tablet, capsule, solution, or thin film form. Further, the composition may comprise any other component disclosed herein. It is also contemplated that the dosage form may be a liquid dosage form which comprises gemfibrozil or gemfibrozil and vitamin A and any other component in the form of a suspension.

In alternative embodiments, the present disclosure further provides an oral cinnamic acid dosage form, which comprises cinnamic acid in buccal, sublingual, tablet, capsule, solution, or thin film form. Further, the composition may comprise any other component disclosed herein. It is also contemplated that the dosage form may be a liquid dosage form which comprises cinnamic acid and any other component in the form of a suspension.

In any of the embodiments of the current invention, the pharmaceutical composition may be in the form of a dosage form wherein the dosage form is either a solid dosage form or a liquid dosage form as described herein.

In any of the embodiments of the current invention, the liquid dosage form may be prepared as a nasal spray or injectable formulation.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE DRAWINGS

FIG. 1 depicts the genotyping of newborn GALC^{-/-} mice was determined by polymerase chain reaction specific for the single base mutation (Functional Biosciences, Wisconsin).

FIG. 2 shows the results for oral treatment with gemfibrozil and the combination of gemfibrozil and vitamin A in protecting myelin in vivo in the cerebellum of GALC^{-/-} mice.

FIG. 3 shows the results for oral treatment with gemfibrozil and the combination of gemfibrozil and vitamin A in protecting myelin in vivo in the corpus callosum of GALC^{-/-} mice.

FIG. 4 depicts additional results for oral treatment with gemfibrozil and the combination of gemfibrozil and vitamin A in protecting myelin in vivo in cerebellum (FIGS. 4A & 4C) and corpus callosum (FIGS. 4B & 4D) of GALC^{-/-} mice.

FIG. 5 shows with luxol fast blue (LFB) staining the protection of myelin by treatment with gemfibrozil and the combination of gemfibrozil and vitamin A in cerebellum (FIG. 5A) and corpus callosum (FIG. 5B) of GALC^{-/-} mice.

FIG. 6 shows the results of oral treatment with gemfibrozil and the combination of gemfibrozil and vitamin A in reducing astroglial activation in vivo in the cerebellum of GALC^{-/-} mice.

FIG. 7 depicts the results of oral treatment with gemfibrozil and the combination of gemfibrozil and vitamin A in reducing the astroglial activation in vivo in the corpus callosum of GALC^{-/-} mice.

FIG. 8 shows that the oral administration of gemfibrozil and the combination of gemfibrozil and vitamin A alleviates motor deficits in GALC^{-/-} mice.

FIG. 9 depicts the results of oral administration of gemfibrozil and the combination of gemfibrozil and vitamin A increasing the lifespan of GALC^{-/-} mice.

FIG. 10 shows the results of administration of oral cinnamic acid in protecting myelin in vivo in the cerebellum of GALC^{-/-} mice.

FIG. 11 depicts the results of oral administration of cinnamic acid in protecting myelin in vivo in the corpus callosum of GALC^{-/-} mice.

FIG. 12 shows further results of the oral administration of cinnamic acid in protecting myelin in vivo in cerebellum (FIGS. 12A & 12C) and corpus callosum (FIGS. 12B & 12D) of GALC^{-/-} mice.

FIG. 13 depicts luxol fast blue (LFB) staining showing protection of myelin in cerebellum (FIG. 13A) and corpus callosum (FIG. 13B) of GALC^{-/-} mice by treatment with cinnamic acid.

FIG. 14 shows the results of oral treatment with cinnamic acid in reducing astroglial activation in vivo in the cerebellum of GALC^{-/-} mice.

FIG. 15 shows the results of oral treatment with cinnamic acid in reducing astroglial activation in vivo in the corpus callosum of GALC^{-/-} mice.

FIG. 16 depicts the results of oral administration of cinnamic acid in reducing the motor deficits in GALC^{-/-} mice.

FIG. 17 depicts the results of oral administration of cinnamic acid in increasing the lifespan of GALC^{-/-} mice.

### DETAILED DESCRIPTION OF THE INVENTION

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The present disclosure provides for compositions and methods for treating various disorders and diseases, particularly, globoid cell leukodystrophy or Krabbe disease (KD). Specifically, the invention provides for methods that include oral administration of gemfibrozil alone, the combination of gemfibrozil and vitamin A or cinnamic acid alone. The inventors have surprisingly found that oral administration of gemfibrozil alone, the combination of gemfibrozil and vitamin A or cinnamic acid alone are capable of protecting myelin, suppressing glial inflammation, improving locomotor activities, and increasing lifespan in an individual or patent afflicted with Krabbe disease.

It is understood that globoid cell leukodystrophy or Krabbe disease (KD) is an inborn error of metabolism leading eventually to an accumulation of the cytotoxic lipid galactosylsphingosine or psychosine in both the central nervous system (CNS) and peripheral nervous system (PNS), and which ultimately leads to demyelination of nerve fibers. Thus the inventors contemplate that since any of the methods or pharmaceutical compositions described herein lead to “protecting myelin,” or “protection of myelin,” that the myelin is “protected” or the “protecting” occurs in either the CNS or PNS, or both, as understood by those of skill in the art.

In various embodiments, the present disclosure provides: i) a method for protecting myelin and inhibiting the progression of KD; ii) methods for suppressing or inhibiting the glial inflammation associated with KD and thus treating or inhibiting the progression of the disease; iii) methods for improving the locomotor activities associated with KD and thus treating or inhibiting the progression of the disease; iv) methods for increasing the lifespan of an individual afflicted with KD and thus treating or inhibiting the progression of the disease; and v) a method for inhibiting progression of a neurodegenerative disorder wherein the neurodegenerative disorder is KD. Such methods comprise administering to a patient in need thereof an effective amount of an oral pharmaceutical composition comprising gemfibrozil or a combination of gemfibrozil and vitamin A. The pharmaceutical composition may be administered to the patient in any including one time per day, two times per day, and three times per day.

In some embodiments, the locomotor activity that is improved by administration of gemfibrozil alone or gemfibrozil in combination with vitamin A may include one or more of walking, running, jumping, and any combination thereof.

The present invention also provides for: i) a method for protecting myelin and inhibiting the progression of KD; ii) methods for suppressing or inhibiting the glial inflammation associated with KD and thus treating or inhibiting the progression of the disease; iii) methods for improving the locomotor activities associated with KD and thus treating or inhibiting the progression of the disease; iv) methods for increasing the lifespan of an individual afflicted with KD and thus treating or inhibiting the progression of the disease; and v) a method for inhibiting progression of a neurodegenerative disorder wherein the neurodegenerative disorder is KD. The methods disclosed comprises administering to a patient in need thereof an effective amount of a oral pharmaceutical composition comprising cinnamic acid.

The disclosed methods contemplate that the pharmaceutical composition comprising cinnamic acid may be administered to the patient in any including one time per day, two times per day, and three times per day.

The disclosed methods also contemplate that the locomotor activity that is improved by administration of cinnamic acid may include one or more of walking, running, jumping, and any combination thereof.

All of the disclosed methods can comprise administering to a patient in need thereof an effective amount of a pharmaceutical composition comprising gemfibrozil alone or gemfibrozil and vitamin A wherein the administering comprises buccal or sublingual administration.

All of the disclosed methods can comprise administering to a patient in need thereof an effective amount of a pharmaceutical composition comprising cinnamic acid wherein the administering comprises buccal or sublingual administration.

Any of the disclosed methods contemplate that the oral gemfibrozil and/or gemfibrozil and vitamin A dosage form, which comprises gemfibrozil or gemfibrozil and vitamin A in buccal, sublingual, tablet, capsule, solution, thin film form. Further, the composition may comprise any other component disclosed herein. It is also contemplated that the dosage form may be a liquid dosage form which comprises gemfibrozil or gemfibrozil and vitamin A and any other component in the form of a suspension.

Any of the disclosed methods contemplate that the oral cinnamic acid dosage form, which comprises cinnamic acid in buccal, sublingual, tablet, capsule, solution, or thin film form. Further, the composition may comprise any other component disclosed herein. It is also contemplated that the dosage form may be a liquid dosage form which comprises cinnamic acid and any other component in the form of a suspension.

In any of the embodiments of the current invention, the pharmaceutical composition may be in the form of a dosage form wherein the dosage form is either a solid dosage form or a liquid dosage form as described herein.

In any of the embodiments of the current invention, the liquid dosage form may be prepared as a nasal spray or injectable formulation.

As used herein, the term “pharmaceutically acceptable carrier” means a non-toxic, inert solid, semi-solid or liquid filler, diluent, encapsulating material or formulation auxiliary of any type.

Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, the contents of which are expressly incorporated herein by reference.

Methods of formulation are well known in the art (see, for example, Remington: s, Mack Publishing Company, Easton, Pa., 19th Edition (1995)). Pharmaceutical compositions for use in accordance with the present disclosure can be in the form of sterile, non-pyrogenic liquid solutions or suspensions, coated capsules, lyophilized powders, or other forms known in the art.

Solid dosage forms for oral administration include, as illustrative but non-limiting examples, capsules, tablets, pills, powders, thin films and granules. In solid dosage forms, the active compound may be mixed with at least one inert, pharmaceutically acceptable excipient or carrier. Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells. Illustrative, non-limiting examples of coatings and shells include enteric coatings and other coatings/shells well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that may be used include, but are not limited to, polymeric substances and waxes.

The active compounds can also be in micro-encapsulated form with one or more excipients as noted above. The solid dosage forms of tablets, dragees, capsules, pills, and granules can be prepared with coatings and shells. The coatings or shells may be, but are not limited to, enteric coatings, release-controlling coatings and other coatings in the pharmaceutical formulating art. In solid dosage forms, the active compound may be admixed with at least one inert diluent. The inert diluent may include, but is not limited to, one or more of, sucrose, lactose or starch. Dosage forms may also comprise additional substances other than inert diluents. The additional substances may be, but are not limited to, tableting lubricants and other tableting aids. The tableting lubricants and other aids may be, but are not limited to, magnesium stearate and microcrystalline cellulose. In the case of capsules, tablets and pills, for example, the dosage forms may also comprise buffering agents. They may comprise opacifying agents. They may be of a composition that releases the active ingredient(s) only, or preferentially, in a certain part of the intestinal tract. The release may be in a delayed manner. Examples of embedding compositions that can be used include, but are not limited to, polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may comprise one or more inert diluents. The inert diluents may be selected from those commonly used in the art. Illustrative, non-limiting examples of inert diluents include water or other solvents, solubilizing agents and emulsifiers (including, but not limited to, ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof). The oral compositions may comprise one or more adjuvants. Illustrative, non-limiting examples of adjuvants include wetting agents, emulsifying and suspending agents, sweetening, flavoring, and perfuming agents.

The liquid dosage forms may be in the form of pharmaceutical suspensions which are understood to be liquid dosage forms containing finely divided insoluble materials (the suspensoid) distributed somewhat uniformly throughout the suspending medium (suspending vehicle) in which the drug exhibits a minimum degree of solubility.

It is also understood that liquid dosage forms may include any dosage form suitable for injection. The pharmaceutical formulations suitable for injectable use, such as, for example, intravenous, subcutaneous, intramuscular and intraperitoneal administration include sterile aqueous solutions or dispersions. In all cases, the form can be sterile and can be fluid to the extent that easy syringeability exists. It can be stable under the conditions of manufacture and storage and can be preserved against the contaminating action of microorganisms such as bacteria and fungi.

In still other embodiments, it is contemplated that the pharmaceutical composition may be part of a thin film administration form. See Karki et al., (2016), “Thin films as an emerging platform for drug delivery,” Asian J Pharmaceutical Sci. 11: pp. 559-574. Generally, it is understood that thin films, alternatively referred as a thin and flexible layer of polymer with or without a plasticizer. Thin films provide a means for targeting sensitive site that may not be possible with tablets or liquid formulations. Thin films have shown the capabilities to improve the onset of drug action, reduce the dose frequency and enhance the drug efficacy.

In yet other embodiments, it is contemplated that the pharmaceutical composition may be in the form of a dosage form that is a transdermal patch consisting of one or more porous membranes covering a reservoir of medication or through body heat melting thin layers of medication embedded in the adhesive.

In some embodiments, effective amounts of the compositions include any amount sufficient to protect myelin and inhibit the progression of KD; ii) suppress or inhibit the glial inflammation associated with KD and thus treat or inhibit the progression of the disease; iii) improve the locomotor activities associated with KD and thus treat or inhibit the progression of the disease; iv) increase the lifespan of an individual afflicted with KD and thus treat or inhibit the progression of the disease; and v) inhibit the progression of a neurodegenerative disorder wherein the neurodegenerative disorder is KD.

The amount of active ingredient, wherein the active ingredient is i) oral gemfibrozil and/or gemfibrozil and vitamin A; or ii) cinnamic acid, that may be combined with the optional carrier materials to produce a single dosage form may vary depending upon the host treated and the particular mode of administration. The specific dose level for any particular patient may depend upon a variety of factors including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

In accordance with certain methods of treatment disclosed in the present application, progression of various disorders is slowed or stopped in a patient (a patient may be a human, a lower mammal, or a warm blooded animal), by administering to the patient an effective amount of the i) oral gemfibrozil and/or gemfibrozil and vitamin A; or ii) cinnamic acid, in such amounts, and for such time as is necessary, to achieve the desired result. An amount of a compound that is effective to slow or stop the progression of a disease or disorder may refer to a sufficient amount of the compound to treat the disease or disorder at a reasonable benefit/risk ratio applicable to any medical treatment.

The total daily usage of the compounds and compositions of the present disclosure may be decided by the attending physician within the scope of sound medical judgment. The specific therapeutically effective dose level for any particular patient may depend upon a variety of factors including the disease or disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; and drugs used in combination or coincidental with the specific compound employed.

The “effective amount” or dose of a compound of the present disclosure, such as i) gemfibrozil and/or gemfibrozil and vitamin A; or ii) cinnamic acid, to be administered to warm-blooded animals (e.g., humans) may vary depending upon the disorder to be treated, the method or mode of drug administration and the desire to minimize any known side effects. In connection with certain neurodegenerative disorders, such as, Krabbe disease or other movement disorders, the effective amount may be from about 1.0 mg/kg to about 5.0 g/kg per day, or any amount or sub-range thereof. In preferred embodiments, the dosage is in the range of about 1.0 mg/kg/day to about 100 mg/kg/day.

The administration may be once per day, twice per day, or more than two times per day. Additionally, in some embodiments, a patient may receive the active ingredients by multiple administration methods including combinations of oral and buccal or sublingual administration. The present disclosure encompasses any combination of the administration techniques described or contemplated herein.

The following examples are intended to illustrate some embodiments of the present disclosure and are not intended to limit the disclosure or scope of the claims in any manner.

### EXAMPLES

### Example 1

Generation of Homozygous GALC^{-/-} Mice

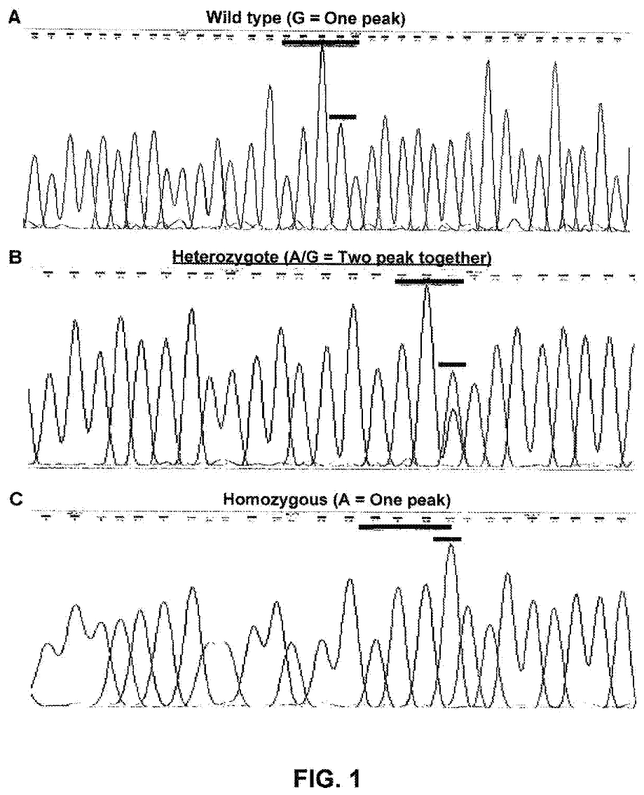

GALC^{+/-} heterozygous mice were purchased commercially and were used for breeding. Briefly, one GALC^{+/-} male and one GALC^{+/-} female mice were kept together in a single cage. After 7 days, the GALC^{-/-} male mouse was separated from the female one. Pups obtained from the female mice were genotyped by polymerase chain reaction specific for the single base mutation to select GALC^{-/-} mice. The results are depicted in FIG. 1.

Heterozygous animals were used only for breeding purposes and were not used in the study. Animal maintaining and experiments were in accordance with National Institute of Health guidelines and were approved by the Institutional Animal Care and Use committee of the Rush University of Medical Center, Chicago, IL. Treated and untreated mice were allowed to survive humanely as long as possible. When a mouse reached a moribund stage, it was sacrificed.

### Example 2

Oral Treatment with Gemfibrozil as Well as the Combination of Gemfibrozil and Vitamin A Protects Myelin In Vivo in the Cerebellum of GALC^{-/-} Mice.

In this example as well as all other examples, since GALC^{-/-} mice die very early, typically around 30 days old, GALC^{-/-} mice were treated with gemfibrozil (8 mg/kg/d) and a combination of gemfibrozil (8 mg/kg/d) and vitamin A (150 IU/kg/d) solubilized in 100 μl 0.5% methylcellulose via gavage starting early, from 10 d of age. Treatment was continued for 15 d. Gemfibrozil (USP grade; Spectrum Chemical) was solubilized in 100 μl 0.5% methyl cellulose (USP grade; Spectrum Chemical) before gavage. Therefore, control GALC^{-/-} mice were also treated with 100 μl 0.5% methyl cellulose as vehicle. To understand the effect of treatment (for comparison purpose), we also included one group of wild type GALC^{+/+} mice. In this and the corresponding examples, the number of mice per group was typically N=6. Since children of both sexes equally affected by Krabbe, both males and females were included in all studies.

As discussed below, after 2 weeks of oral feeding, several parameters were monitored including locomotor activity followed by examining myelin status and glial activation in cerebellum and corpus callosum. Longevity of GALC^{-/-} mice after drug treatment was also examined. In that case, GALC^{-/-} mice were treated with the drugs daily until mice reached the moribund stage.

Monitoring Myelination Status:

Levels of myelin basic protein (MBP) and proteolipid protein (PLP) in cerebellum and corpus callosum were examined by Western blot as described previously. See for example: Chandra et al., (2017) J Immunol 198, 4312-4326; Chandra et al., (2019) J Alzheimers Dis Rep 3, 149-168; Chandra et al., (2019) Neurobiol Dis 124, 379-395. Briefly, samples were homogenized in RIPA buffer containing protease and phosphatase inhibitors (Sigma), rotated end over end for 30 min at 4° C. and centrifuged for 10 min at 15,000 g. The supernatant was aliquoted and stored at −80° C. until use. Protein concentrations were determined using the BCA assay (Thermo Fisher), and 15-30 μg sample was heat-denatured and resolved on 10% or 12% polyacrylamide-SDS gels in MES buffer (50 mM MES, 50 mM Tris base, 0.1% SDS, 1 mM EDTA, pH 7.3) or 1×SDS Running Buffer. Proteins were transferred to 0.45 μm nitrocellulose membranes in Towbin Buffer (25 mM Tris, 192 mM glycine, 20% (w/v) methanol) under wet conditions (40 V for 120 mins). Membranes were blocked for 1 h with blocking buffer (Li-Cor), incubated with primary antibodies overnight at 4° C. under shaking conditions, washed, incubated with IR-dye labeled secondary antibodies for 45 min at room temperature, washed and visualized with the Odyssey Infrared Imaging System (Li-Cor). Blots were converted to binary, analyzed using Image J and normalized to the loading control (Actin).

Immunofluorescence Analysis for PLP:

After 2 weeks of treatment, mice were anesthetized and perfused with PBS (pH 7.4) and then with 4% (w/v) paraformaldehyde solution in PBS. See for example: Khasnavis et al., (2013) J Neuroimmune Pharmacol 9, 218-232; Ghosh et al., (2007) Proc Natl Acad Sci USA 104, 18754-18759; Patel et al., (2018) Proc Natl Acad Sci USA 115, E7408-E7417. Briefly, brains were incubated in PBS containing 0.05% Tween 20 (PBST) and 10% sucrose for 3 h and then 30% sucrose overnight at 4° C. Hemisected brains was then embedded in O.C.T (Tissue Tech) at −80° C. and processed for conventional cryosectioning. Frozen cerebellar and corpus callosum sections (40 micron thick) were treated with cold ethanol (−20° C.) followed by two rinses in PBS, blocking with 3% bovine serum albumin in PBST and labeling with anti-PLP antibody. After three washes in PBST, sections were further incubated with Cy5 (Jackson ImmunoResearch Laboratories, Inc.). The samples were mounted and observed under the Olympus BX41 fluorescent microscope equipped with a Hamamatsu ORCA-03G camera. Captured images were calibrated with the scale bar and then opened in ImageJ software for further quantification analysis. For measuring PLP MFI, a closed square tool was used to draw the boundary around PLP-ir signals followed by monitoring MFI using ImageJ software. The final MFI was analyzed after subtracting the value with the background signal of respective images.

Staining for Myelin:

Cerebellar and corpus callosum sections of paraformaldehyde-fixed brain tissues were stained with Luxol fast blue for myelin as described elsewhere. See for example: Mondal, et al., (2017) J Clin Cell Immunol 8; Mondal et al., (2018) Sci Signal 11; Mondal et al., (2020) Proc Natl Acad Sci USA 117, 21557-21567.

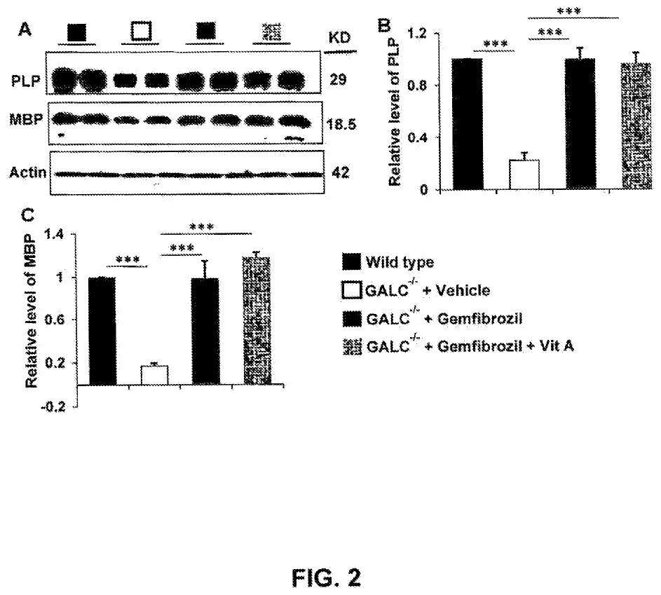

FIG. 2 depicts the results where ten days old GALC^{-/-} mice were fed with gemfibrozil (8 mg/Kg/day) and a combination of gemfibrozil (8 mg/Kg/day) and vitamin A (150 IU/Kg/day) for 15 days followed by monitoring the levels of PLP and MBP in cerebellum by Western blot (A). Actin was run as loading control. Bands were scanned and values (B, PLP/Actin; C, MBP/Actin) presented as relative to control. Results were analyzed by one-way ANOVA followed by Dunnett's multiple comparison tests; ***p<0.001. Data are represented as mean±SEM of 4 mice per group. FIG. 2 shows that the levels of PLP and MBP were significantly increased in mice receiving oral treatment with gemfibrozil or the combination of gemfibrozil and vitamin A indicating that such treatment protects myelin in vivo in the cerebellum of GALC^{-/-} mice.

### Example 3

Oral Treatment with Gemfibrozil and the Combination of Gemfibrozil and Vitamin A Protects Myelin In Vivo in the Corpus Callosum of GALC^{-/-} Mice.

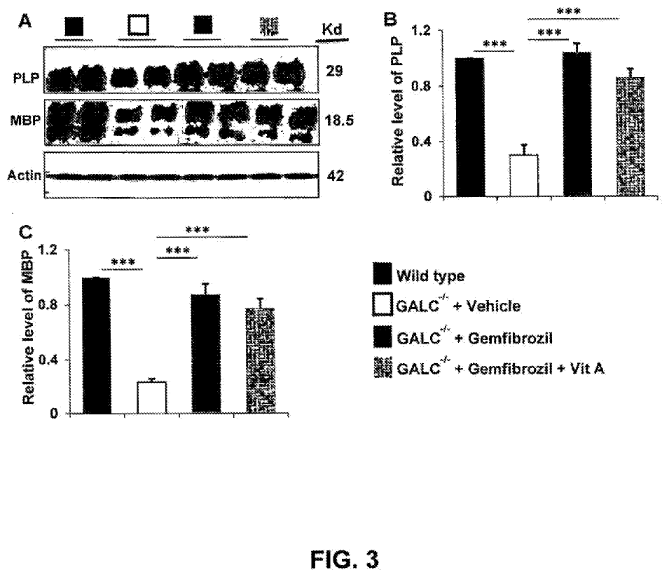

Mice received oral treatment for 15 days with gemfibrozil or the combination of gemfibrozil and vitamin A as in Example 1. Levels of PLP and MBP were monitored in the corpus callosum by Western blot (A). Actin was run as loading control. Bands were scanned and values (B, PLP/Actin; C, MBP/Actin) presented as relative to control. Results were analyzed by one-way ANOVA followed by Dunnett's multiple comparison tests; ***p<0.001. Data are represented as mean±SEM of 4 mice per group. FIG. 3 shows that the levels of PLP and MBP were significantly increased in mice receiving oral treatment with gemfibrozil or the combination of gemfibrozil and vitamin A.

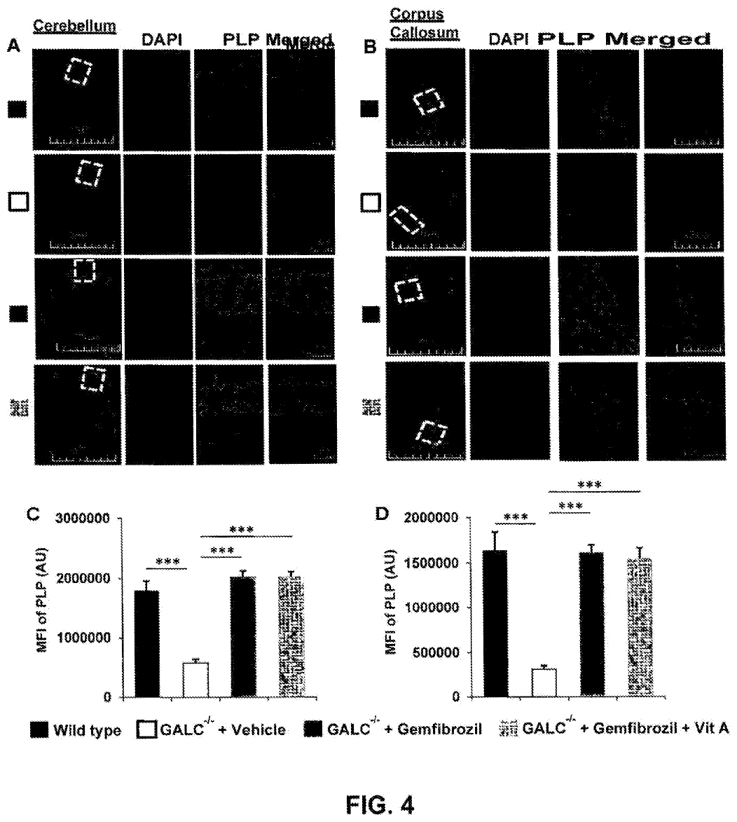

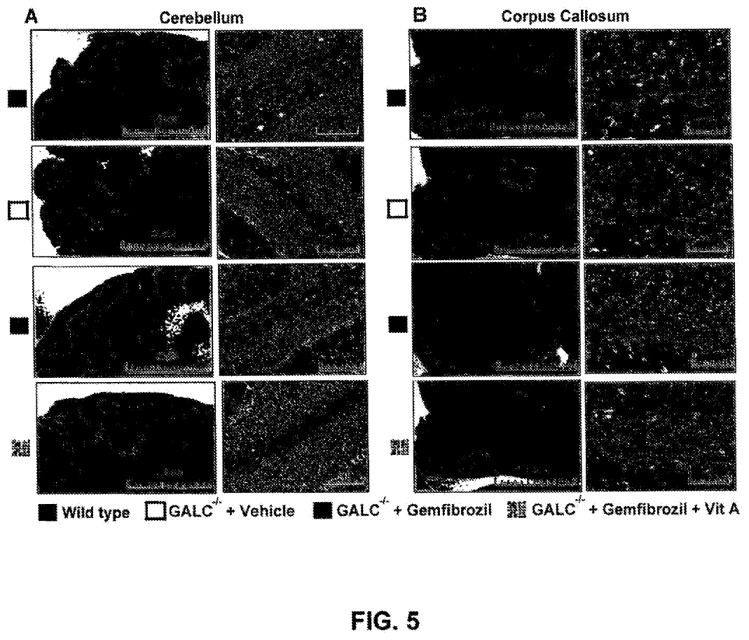

FIG. 4 shows similar results following immunostaining of cerebellar (A) and corpus callosum (B) sections with PLP following identical treatment. DAPI was used to visualize nucleus. Mean fluorescence intensity (MFI) of PLP was quantified (C, cerebellum; D, corpus callosum) in two different sections of each of five mice (n=5) per group. Results were analyzed by one-way ANOVA followed by Dunnett's multiple comparison tests; ***p<0.001. FIG. 5 similarly depicts luxol fast blue (LFB) staining of myelin in the cerebellum (A) and corpus callosum (B) of GALC^{-/-} mice following identical treatment. Results represent analysis of two different sections of each of five mice (n=5) per group.

The results from Examples 1 and 2 indicate that oral treatment with gemfibrozil or the combination of gemfibrozil and vitamin A protects myelin in vivo in the cerebellum and corpus callosum of GALC^{-/-} mice.

### Example 4

Oral Treatment with Gemfibrozil or the Combination of Gemfibrozil and Vitamin A Reduces Astroglial Activation In Vivo in the Cerebellum of GALC^{-/-} Mice.

Monitoring Glial Activation:

During activation, glial cells express inducible nitric oxide synthase (iNOS) to produce nitric oxide. Therefore, to monitor astroglial activation, in Examples 3 and 4, the level of glial fibrillary acidic protein (GFAP), a marker of astroglia, and iNOS in cerebellum and corpus callosum was examined by Western blot as described previously. See for example: Chandra et al., (2017) J Immunol 198, 4312-4326; Chandra et al., (2019) J Alzheimers Dis Rep 3, 149-168; Chandra et al., (2019) Neurobiol Dis 124, 379-395.

Briefly, samples were homogenized in RIPA buffer containing protease and phosphatase inhibitors (Sigma), rotated end over end for 30 min at 4° C. and centrifuged for 10 min at 15,000 g. The supernatant was aliquoted and stored at −80° C. until use. Protein concentrations were determined using the BCA assay (Thermo Fisher), and 15-30 μg sample was heat-denatured and resolved on 10% or 12% polyacrylamide-SDS gels in MES buffer (50 mM MES, 50 mM Tris base, 0.1% SDS, 1 mM EDTA, pH 7.3) or 1×SDS Running Buffer. Proteins were transferred to 0.45 μm nitrocellulose membranes in Towbin Buffer (25 mM Tris, 192 mM glycine, 20% (w/v) methanol) under wet conditions (40 V for 120 mins). Membranes were blocked for 1 h with blocking buffer (Li-Cor), incubated with primary antibodies overnight at 4° C. under shaking conditions, washed, incubated with IR-dye labeled secondary antibodies for 45 min at room temperature, washed and visualized with the Odyssey Infrared Imaging System (Li-Cor). Blots were converted to binary, analyzed using Image J and normalized to the loading control (Actin).

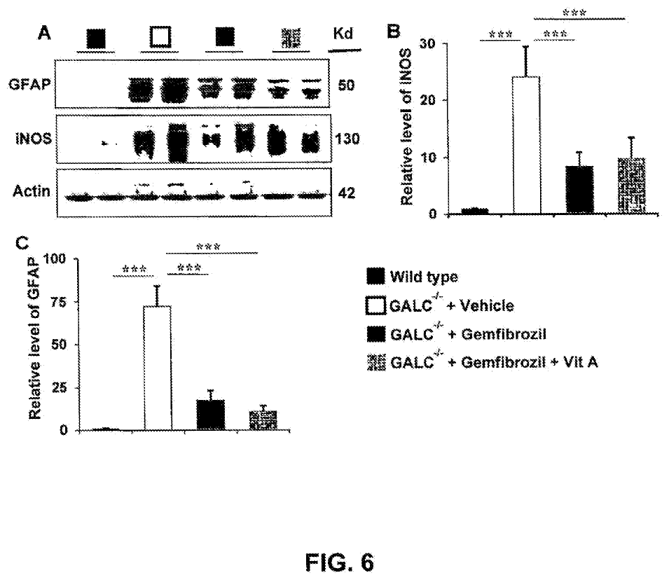

Mice received oral treatment with gemfibrozil or the combination of gemfibrozil and vitamin A as above. Following 15 days of treatment, levels of glial fibrillary acidic protein (GFAP) and inducible nitric oxide synthase (iNOS) were monitored in the cerebellum by Western blot (A). Actin was run as loading control. Bands were scanned and values (B, iNOS/Actin; C, GFAP/Actin) presented as relative to control. Results were analyzed by one-way ANOVA followed by Dunnett's multiple comparison tests; ***p<0.001. Data are represented as mean±SEM of 4 mice per group. FIG. 6 shows that drug treatment significantly reduced the levels of iNOS and GFAP in the cerebellum of GALC^{-/-} mice.

### Example 5

Oral Treatment with Gemfibrozil or the Combination of Gemfibrozil and Vitamin A Reduces Astroglial Activation In Vivo in the Corpus Callosum of GALC^{-/-} Mice.

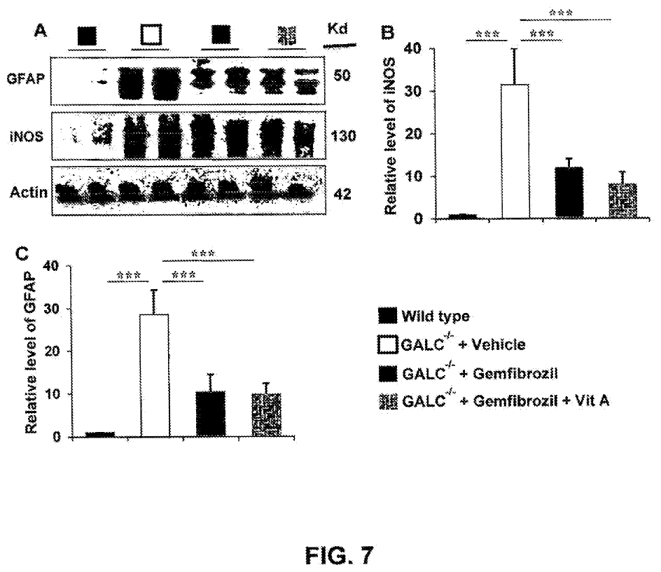

Mice received oral treatment with gemfibrozil or the combination of gemfibrozil and vitamin A as above. Following 15 days of treatment, levels of glial fibrillary acidic protein (GFAP) and inducible nitric oxide synthase (iNOS) were monitored in the cerebellum by Western blot (A). Actin was run as loading control. Bands were scanned and values (B, iNOS/Actin; C, GFAP/Actin) presented as relative to control. Results were analyzed by one-way ANOVA followed by Dunnett's multiple comparison tests; ***p<0.001. Data are represented as mean±SEM of 4 mice per group. FIG. 7 shows that drug treatment significantly reduced the levels of iNOS and GFAP in the corpus callosum of GALC^{-/-} mice.

The results from Examples 3 and 4 indicate that oral treatment with gemfibrozil or the combination of gemfibrozil and vitamin A reduces astroglial activation in vivo in the cerebellum and corpus callosum of GALC^{-/-} mice.

### Example 6

Oral Gemfibrozil and the Combination of Gemfibrozil and Vitamin a Alleviates Motor Deficits in GALC^{-/-} Mice.

Locomotor Activity:

The open field test was conducted for analyzing general motor activities of mice. Locomotor activities were monitored by open field performance and footprint analysis as described in previous studies. See for example: Patel, et al., (2019) J Neuroimmune Pharmacol 14, 503-518; Patel et al., (2018) Proc Natl Acad Sci USA 115, E7408-E7417; Rangasamy, et al., (2018) J Clin Invest 128, 4297-4312. Mice were placed in the center of a square wooden open field arena (40×40 cm, 30 cm high walls) and allowed to explore freely for 5 min. The movements of the mice were recorded using a camera linked to the Noldus system and EthoVisionXT software. Several parameters including velocity, total distance moved, movement/center point frequency, body elongation, etc. were analyzed to assess the general locomotor activity.

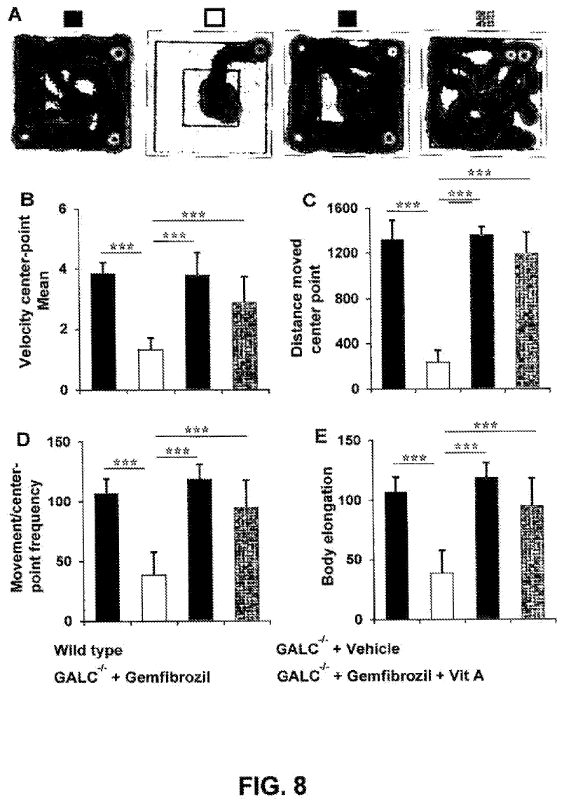

Mice received oral treatment with gemfibrozil or the combination of gemfibrozil and vitamin A as above. Following 15 days of treatment, open field locomotor activities were measured. The results are depicted in FIG. 8. Heatmaps demonstrate the horizontal locomotor activities of experimental animals in the open field arena as captured by the Noldus software (A). Parameters related to movement of animals were obtained from the software and presented as velocity center point (B), distance moved center point (C), movement-center point frequency (D), and body elongation (E). Statistics was conducted using one-way ANOVA followed by Dunnett's multiple comparison tests; ***p<0.001. Data are represented as mean±SEM of 6 mice per group. Motor activity is increased in drug treated animals versus vehicle treatment.

The results indicate that oral treatment with gemfibrozil or the combination of gemfibrozil and vitamin A alleviates the motor deficits in GALC^{-/-} mice.

### Example 7

Oral Gemfibrozil and the Combination of Gemfibrozil and Vitamin a Increases the Lifespan of GALC^{-/-} Mice.

Mice received oral treatment with gemfibrozil or the combination of gemfibrozil and vitamin A as above. Mice were treated continually until they reached the moribund stage at which point they were humanely sacrificed.

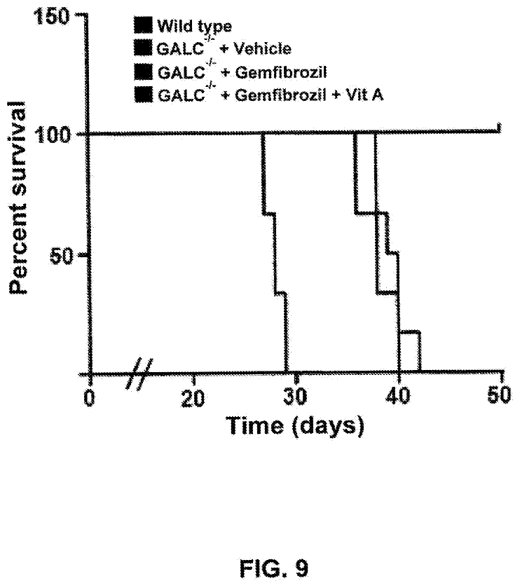

FIG. 9 shows the results of the percentage of survival is shown by Kaplan-Meier plot. The data demonstrate that oral treatment with gemfibrozil or the combination of gemfibrozil and vitamin A increase the lifespan of GALC^{-/-} mice.

### Example 8

Oral Cinnamic Acid Protects Myelin In Vivo in the Cerebellum of GALC^{-/-} Mice.

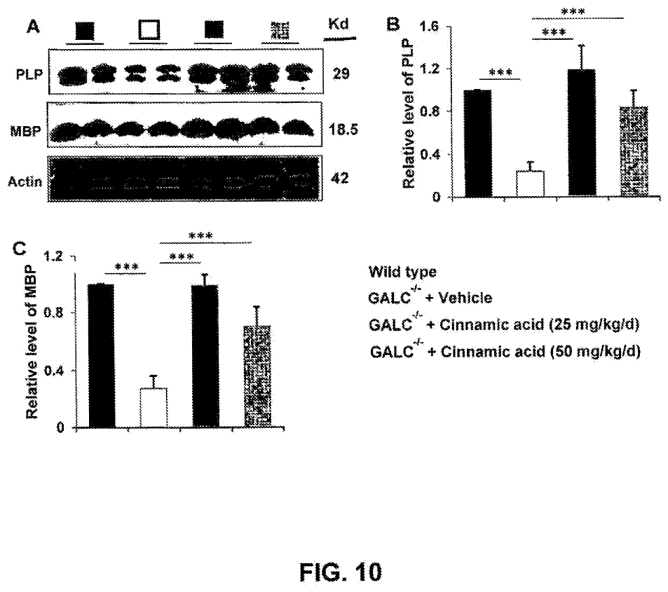

Mice (10 d old) received were fed with 25 and 50 mg/kg/day of cinnamic acid for 15 days. Levels of PLP and MBP were monitored in the cerebellum by Western blot (A). Actin was run as loading control. Bands were scanned and values (B, PLP/Actin; C, MBP/Actin) presented as relative to control. Results were analyzed by one-way ANOVA followed by Dunnett's multiple comparison tests; ***p<0.001. Data are represented as mean±SEM of 4 mice per group. FIG. 10 shows that the levels of PLP and MBP were significantly increased in mice receiving oral treatment with oral cinnamic acid.

### Example 9

Oral Cinnamic Acid Protects Myelin In Vivo in the Corpus Callosum of GALC^{-/-} Mice.

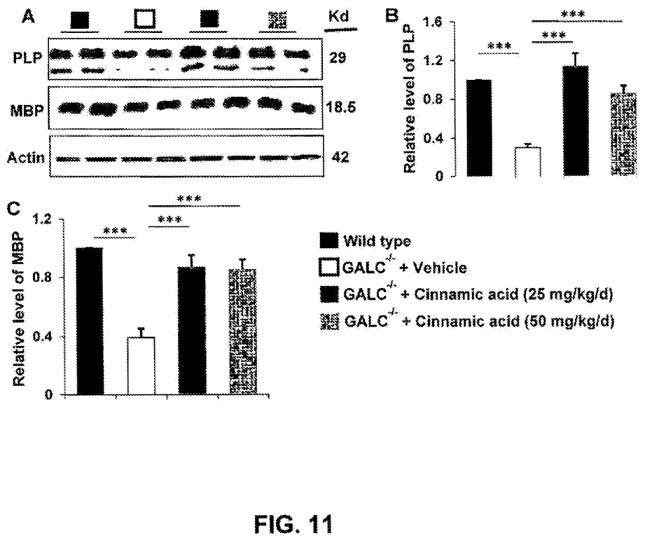

Mice received oral treatment for 15 days with cinnamic acid as described above in Example 7. Levels of PLP and MBP were monitored in the corpus callosum by Western blot (A). Actin was run as loading control. Bands were scanned and values (B, PLP/Actin; C, MBP/Actin) presented as relative to control. Results were analyzed by one-way ANOVA followed by Dunnett's multiple comparison tests; ***p<0.001. Data are represented as mean±SEM of 4 mice per group. FIG. 11 shows that the levels of PLP and MBP were significantly increased in mice receiving oral cinnamic acid.

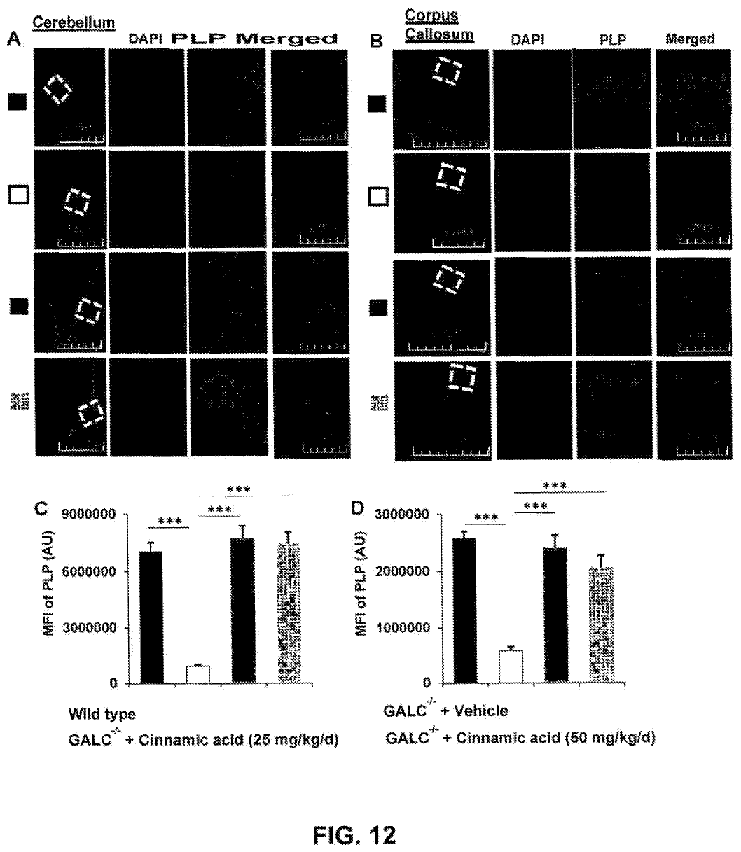

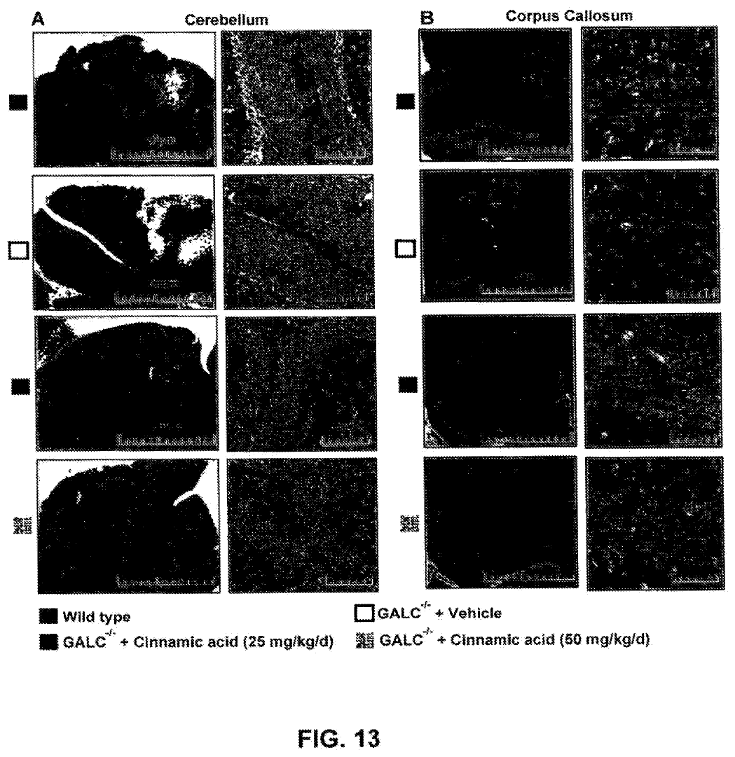

FIG. 12 shows similar results following immunostaining of cerebellar (A) and corpus callosum (B) sections with PLP following identical treatment. DAPI was used to visualize nucleus. Mean fluorescence intensity (MFI) of PLP was quantified (C, cerebellum; D, corpus callosum) in two different sections of each of five mice (n=5) per group. Results were analyzed by one-way ANOVA followed by Dunnett's multiple comparison tests; ***p<0.001. FIG. 13 similarly depicts luxol fast blue (LFB) staining of myelin in the cerebellum (A) and corpus callosum (B) of GALC^{-/-} mice following identical treatment. Results represent analysis of two different sections of each of five mice (n=5) per group.

### Example 10

Oral Cinnamic Acid Reduces Astroglial Activation In Vivo in the Cerebellum of GALC^{-/-} Mice.

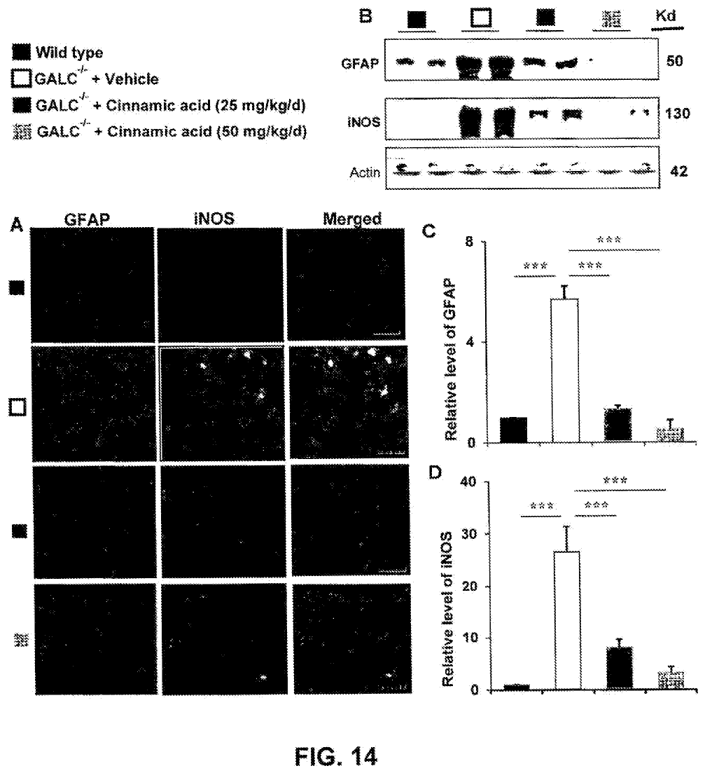

Mice received oral treatment with cinnamic acid as described in Example 7. Glial activation was monitored as in Examples 3 and 4. Following 15 days of treatment, levels of glial fibrillary acidic protein (GFAP) and inducible nitric oxide synthase (iNOS) were monitored in the cerebellum by Western blot (A). Actin was run as loading control. Bands were scanned and values (B, iNOS/Actin; C, GFAP/Actin) presented as relative to control. Results were analyzed by one-way ANOVA followed by Dunnett's multiple comparison tests; ***p<0.001. Data are represented as mean±SEM of 4 mice per group. FIG. 14 shows that drug treatment significantly reduced the levels of iNOS and GFAP in the cerebellum of GALC^{-/-} mice.

### Example 11

Oral Cinnamic Acid Reduces Astroglial Activation In Vivo in the Corpus Callosum of GALC^{-/-} Mice.

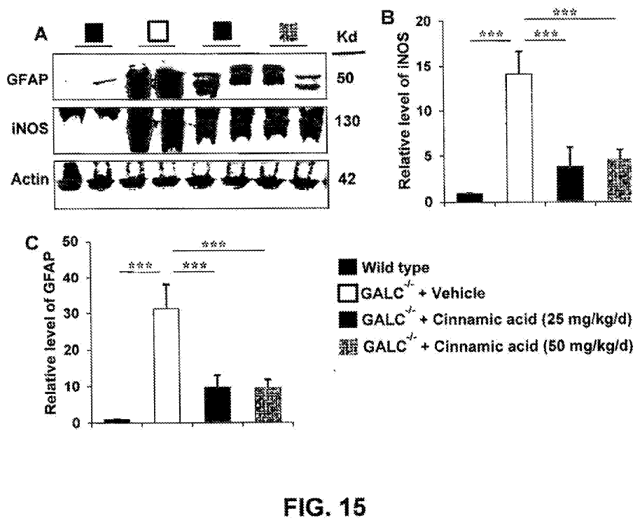

Mice received oral treatment with cinnamic acid as described in Example 7. Glial activation was monitored as in Examples 3 and 4. Following 15 days of treatment, levels of glial fibrillary acidic protein (GFAP) and inducible nitric oxide synthase (iNOS) were monitored in the corpus callosum by Western blot (A). Actin was run as loading control. Bands were scanned and values (B, iNOS/Actin; C, GFAP/Actin) presented as relative to control. Results were analyzed by one-way ANOVA followed by Dunnett's multiple comparison tests; ***p<0.001. Data are represented as mean±SEM of 4 mice per group. FIG. 15 shows that drug treatment significantly reduced the levels of iNOS and GFAP in the corpus callosum of GALC^{-/-} mice.

The results from Examples 8 and 9 indicate that oral treatment with cinnamic acid reduces astroglial activation in vivo in the cerebellum and corpus callosum of GALC^{-/-} mice.

### Example 12

Oral Cinnamic Acid Alleviates Motor Deficits in GALC^{-/-} Mice.

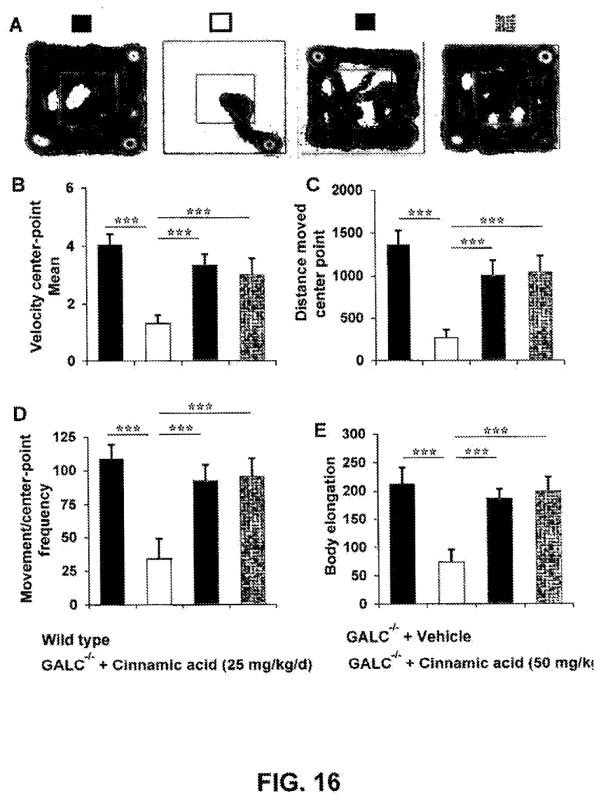

Mice received oral treatment with cinnamic acid as described in Example 7. Locomotor activity was monitored as in Example 5. Following 15 days of treatment, open field locomotor activities were measured. The results are depicted in FIG. 16. Heatmaps demonstrate the horizontal locomotor activities of experimental animals in the open field arena as captured by the Noldus software (A). Parameters related to movement of animals were obtained from the software and presented as velocity center point (B), distance moved center point (C), movement-center point frequency (D), and body elongation (E). Statistics was conducted using one-way ANOVA followed by Dunnett's multiple comparison tests; ***p<0.001. Data are represented as mean±SEM of 6 mice per group. Motor activity is increased in drug treated animals versus vehicle treatment.

The results indicate that oral treatment with cinnamic acid alleviates the motor deficits in GALC^{-/-} mice.

### Example 13

Oral Cinnamic Acid Increases the Lifespan of GALC^{-/-} Mice.

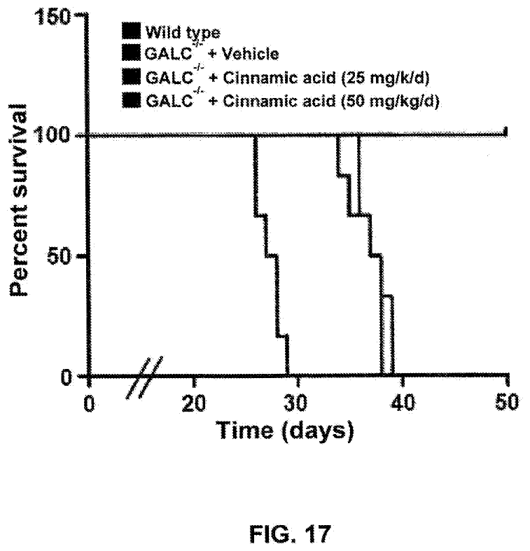

Mice received oral treatment with cinnamic acid as described in Example 7 and lifespan monitored as in Example 6. FIG. 17 shows the results of the percentage of survival is shown by Kaplan-Meier plot. The data demonstrate that oral treatment with cinnamic acid increases the lifespan of GALC^{-/-} mice.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

1. A method for protecting the degeneration of myelin in the nervous system of a patient with globoid cell leukodystrophy or Krabbe disease, comprising the administration to a patient in need thereof an effective amount of a pharmaceutical composition comprising gemfibrozil and at least one pharmaceutically acceptable excipient or carrier.

2. The method of claim 1, wherein the pharmaceutical composition further comprises vitamin A.

3. The method of claim 1, wherein the administration of the pharmaceutical composition inhibits the progression of the globoid cell leukodystrophy or Krabbe disease.

4. A method for suppressing glial inflammation in the brain of a patient with globoid cell leukodystrophy or Krabbe disease, comprising the administration to a patient in need thereof an effective amount of a pharmaceutical composition comprising gemfibrozil and at least one pharmaceutically acceptable excipient or carrier.

5. The method of claim 5, wherein the pharmaceutical composition further comprises vitamin A.

6. The method of claim 5, wherein the administration of the pharmaceutical composition inhibits the progression of the globoid cell leukodystrophy or Krabbe disease.

7. A method for improving the locomotor activity of a patient with globoid cell leukodystrophy or Krabbe disease, comprising the administration to a patient in need thereof an effective amount of a pharmaceutical composition comprising gemfibrozil and at least one pharmaceutically acceptable excipient or carrier.

8. The method of claim 7, wherein the pharmaceutical composition further comprises vitamin A.

9. The method of claim 7, wherein the administration of the pharmaceutical composition inhibits the progression of the globoid cell leukodystrophy or Krabbe disease.

10. A method for increasing the lifespan of a patient with globoid cell leukodystrophy or Krabbe disease, comprising the administration to a patient in need thereof an effective amount of a pharmaceutical composition comprising gemfibrozil and at least one pharmaceutically acceptable excipient or carrier.

11. The method of claim 9, wherein the pharmaceutical composition further comprises vitamin A.

12. The method of claim 9, wherein the administration of the pharmaceutical composition inhibits the progression of the globoid cell leukodystrophy or Krabbe disease.

13. A method of treating a patient with globoid cell leukodystrophy or Krabbe disease, comprising the administration to a patient in need thereof an effective amount of a pharmaceutical composition comprising gemfibrozil alone or in combination with vitamin A and at least one pharmaceutically acceptable excipient or carrier.

14. The method of claim 13, wherein the administration of the pharmaceutical composition inhibits the progression of the globoid cell leukodystrophy or Krabbe disease.

15. The method of claim 1, wherein the pharmaceutical composition is administered to the patient one time per day, two times per day, or three times per day.

16. A method of treating or inhibiting progression of a neurodegenerative disorder wherein the neurodegenerative disorder is globoid cell leukodystrophy or Krabbe disease and wherein the method comprises administering to a patient in need thereof an effective amount of a pharmaceutical composition comprising gemfibrozil alone or gemfibrozil in combination with vitamin A and at least one pharmaceutically acceptable excipient or carrier.

17. A method for protecting the degeneration of myelin in the nervous system of a patient with globoid cell leukodystrophy or Krabbe disease, comprising the administration to a patient in need thereof an effective amount of a pharmaceutical composition comprising cinnamic acid and at least one pharmaceutically acceptable excipient or carrier.

18. The method of claim 17, wherein the administration of the pharmaceutical composition inhibits the progression of the globoid cell leukodystrophy or Krabbe disease.

19. A method for suppressing glial inflammation in the brain of a patient with globoid cell leukodystrophy or Krabbe disease, comprising the administration to a patient in need thereof an effective amount of a pharmaceutical composition comprising cinnamic acid and at least one pharmaceutically acceptable excipient or carrier.

20. The method of claim 19, wherein the administration of the pharmaceutical composition inhibits the progression of the globoid cell leukodystrophy or Krabbe disease.

21. A method for improving the locomotor activity of a patient with globoid cell leukodystrophy or Krabbe disease, comprising the administration to a patient in need thereof an effective amount of a pharmaceutical composition comprising cinnamic acid and at least one pharmaceutically acceptable excipient or carrier.

22. The method of claim 21, wherein the administration of the pharmaceutical composition inhibits the progression of the globoid cell leukodystrophy or Krabbe disease.

23. A method for increasing the lifespan of a patient with globoid cell leukodystrophy or Krabbe disease, comprising the administration to a patient in need thereof an effective amount of a pharmaceutical composition comprising cinnamic acid and at least one pharmaceutically acceptable excipient or carrier.

24. The method of claim 23, wherein the administration of the pharmaceutical composition inhibits the progression of the globoid cell leukodystrophy or Krabbe disease.

25. A method of treating a patient with globoid cell leukodystrophy or Krabbe disease, comprising the administration to a patient in need thereof an effective amount of a pharmaceutical composition comprising cinnamic acid and at least one pharmaceutically acceptable excipient or carrier.

26. The method of claim 25, wherein the administration of the pharmaceutical composition inhibits the progression of the globoid cell leukodystrophy or Krabbe disease.

27. The method of claim 16, wherein the pharmaceutical composition is administered to the patient one time per day, two times per day, or three times per day.

28. A method of treating or inhibiting progression of a neurodegenerative disorder wherein the neurodegenerative disorder is globoid cell leukodystrophy or Krabbe disease and wherein the method comprises administering to a patient in need thereof an effective amount of a pharmaceutical composition comprising cinnamic acid and at least one pharmaceutically acceptable excipient or carrier.

29. The method of claim 1, wherein the pharmaceutical composition is selected from oral solid dosage and liquid dosage forms.

30. The method of claim 29, wherein the oral solid dosage is one or more of oral, buccal or sub-lingual.

31. The method of claim 1, wherein the pharmaceutical composition is a suspension.
